(19) United States
(12) Patent Application Publication
(10) Pub. No.: US 2019/0039048 A1
(43) Pub. Date: Feb. 7, 2019

(54) ACTIVATED PHYLLOSILICATE CLAY OXIDATION CATALYST

(71) Applicant: UNIVERSITY OF SOUTHERN CALIFORNIA, Los Angeles, CA (US)

(72) Inventors: Brent MELOT, Los Angeles, CA (US); Erica HOWARD, Los Angeles, CA (US)

(21) Appl. No.: 16/055,993

(22) Filed: Aug. 6, 2018

Related U.S. Application Data
(60) Provisional application No. 62/541,450, filed on Aug. 4, 2017.

(51) Int. Cl. B01J 21/16; B01J 6/00

(52) U.S. Cl. CPC B01J 21/16; C07C 51/23; B01J 6/001

(57) ABSTRACT

A method is disclosed for the activation of transition-metal containing phyllosilicate structures and uses of the activated phyllosilicates. The process of activation either liberates a proton or an entire hydroxyl group from the structure, creating a material with a mixed-valence state that can oxidize alcohols to aldehydes and ketones.

### RELATED APPLICATIONS

This application claims priority under 35 U.S.C. § 119(e) to U.S. Provisional Patent Application No. 62/541,450, filed Aug. 4, 2017, which is incorporated herein by reference.

### BACKGROUND OF THE INVENTION

Low-cost and sustainable oxidation catalysts play a pivotal role in decreasing society's reliance on fossil fuels and providing ways to produce commodity chemicals in a greener way. Specifically, selective oxidation catalysts can be used to create key chemicals and intermediates like alcohols, aldehydes and ethers. These oxidation reactions represent a promising avenue for creating value added chemicals from biomass.

Heterogeneous catalysts are an innovative alternative for enabling sustainable chemistry due to their inherent practical advantages concerning catalyst stability, separation, and reuse. Prerequisite for the rational design of solid catalysts is a proper understanding of the structure of the active sites as well as of the mechanism of the chemical transformations occurring on the catalytic surface. Steering catalysis toward a desired pathway requires sophisticated strategies for controlling the environment at the catalytically active sites. Accordingly, there is a need for new heterogeneous catalysts for greener chemical transformations. There is also a need for heterogeneous catalysts that have high selectivity, high catalyst stability, and that can be readily separated and reused.

### SUMMARY

The invention provides a low cost, Earth-abundant, sustainable heterogeneous catalyst for the oxidation of alcohols at significantly low temperatures, for example, at room temperature to about approximately the boiling point of the substrate. Additionally, a method is disclosed for activating catalytic properties of transition-metal containing phyllosilicate clays. Depending on the metal content, the process of activation either liberates a proton, an entire hydroxyl group, or both, from the structure of the clay, creating a material with a mixed-valence state that can be used for oxidizing alcohols, for example, to aldehydes or ketones.

Accordingly, the invention provides a heterogeneous catalyst comprising crystallized phyllosilicate structure and mixed oxidation states of one or more metals in the phyllosilicate structure. The catalyst can be in an activated catalyst form such that protons, hydroxyls, or both, have been removed from the surface of the catalyst compared to the non-activated catalyst form.

In one embodiment, the catalyst comprises an activated form of the phyllosilicate M₂X₄O₁₀(OH)₂.4H₂O; AM₂X₄O₁₀(OH)₂.4H₂O; M₃X₂O₅(OH)₄; M₂X₂O₅(OH)₄; or A₂M₃X₄O₁₀(OH)₈. The phyllosilicates can be smectite, mica, serpentine, kaolinite, or chlorite types of phyllosilicates.

In various embodiments, M is Sc, Ti, V, Cr, Mn, Fe, Co, Ni, Cu, Zn, Mg or Al, or solid solutions thereof, wherein the catalyst comprises M moieties in the 2+ oxidation state, in the 3+ oxidation state, or both.

A can be Li, Na, K, Rb, Cs, NH₄, Mg, Ca, Sr, or Ba, or solid solutions thereof; and

X can Fe, Al, Si, or Ti, or solid solutions thereof.

The catalyst can have phyllosilicate tetrahedral sheets, octahedral sheets, or 2:1 tetrahedral:octahedral layers.

In one specific embodiment, M is Ni and no other metals are present in the phyllosilicate catalyst structure.

The catalyst in activated form, where protons, hydroxyls, or both, have been removed from the surface of the catalyst compared to the non-activated catalyst form, can be indicated by a formula having less than the original stoichiometric amount of protons or hydroxyl groups. For example, when the formula of the phyllosilicate is M₃X₂O₅(OH)₄, the activated form is shown as M₃X₂O₅(OH)₄₋ₓ, where x is greater than 0 and less than or equal to 3. In some embodiments, x is 0.5 to about 3, about 1 to about 3, about 0.5 to about 2, about 1 to about 2, and often about 1.2.

In some embodiments, the catalyst comprises Ni₃Si₂O₅(OH)₄₋ₓ, where x is 1, 2, or 3, or any fraction in between the recited integers.

In various embodiments, the ratio of OH:O ions on catalyst surface is less than 1:1, or less than 1:1.5. For example, the ratio of OH:O ions on catalyst surface can be less than 1:2.

In one specific embodiment, the catalyst comprises platelet structures.

The invention also provides a method to oxidize an alcohol moiety of an organic compound comprising contacting an organic compound that comprises an alcohol moiety with an effective amount of a catalyst described herein, and a base, at a temperature sufficient to oxidize the alcohol moiety. The alcohol moiety can be oxidized to an aldehyde, or a ketone, and optionally in some instances, to a carboxylic acid.

The oxidation can be carried out at any suitable and effective temperature below the boiling point of the chosen solvent or substrate, often about 20° C. to about 90° C. for organic compounds having an aliphatic alcohol. The oxidation can be carried out under an oxygen-containing atmosphere, such as under air, or under an atmosphere of oxygen, optionally a pressurized atmosphere of oxygen, each optionally after evacuation of the reaction headspace of other gasses. The base can be any suitable and effective base, such as an alkali metal hydroxide (e.g., LiOH, NaOH, or KOH), or an alkaline earth metal hydroxide (e.g., Mg(OH)₂ or Ca(OH)₂).

The invention further provides a method to produce H₂ gas comprising contacting an organic compound that comprises a hydroxyl with an effective amount of a catalyst described herein, and a base, at a temperature sufficient to produce H₂ gas. The production of H₂ gas can be concurrent with dehydrogenation, and optionally, subsequent decarboxylation, of the organic compound.

The invention additionally provides a method for preparing an oxidation catalyst comprising pre-calcining a phyllosilicate catalyst precursor in a pre-calcining zone in air at a temperature of less than about 600° C. to form an activated form of the starting material, which form comprises mixed-valence states of metals in the phyllosilicate. The phyllosilicate catalyst precursor can be activated through thermal or chemical treatment to produce a composition with mixed oxidation states on the metals. The activation can involve the loss of a proton, the loss of a hydroxyl, or a combination thereof, from the surface of the oxidation catalyst. More than 20%, more than 25%, or more than 30% of protons attached to hydroxyls and/or the hydroxyls on the surface of the catalyst can be removed by the pre-calcining. The pre-calcining can be carried out at any suitable and effective temperature, such as about 400° C. to about 590° C., about 450° C. to about 550° C., or about 500° C.

In another embodiment, the invention provides method for preparing an oxidation catalyst, the method comprising: (a) preparing a transition-metal containing phyllosilicate catalyst precursor; and (b) pre-calcining the catalyst precursor in a pre-calcining zone in air at a temperature of less than 600° C., thereby activating the transition-metal containing phyllosilicate to form a partially deprotonated form of the starting material, which form is stable at ambient temperatures (e.g., at about 22° C.).

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. The building blocks of a typical phyllosilicate structure: (a) and (b) tetrahedral sheet, (c) and (d) octahedral sheet, (e) 2:1 tetrahedral:octahedral layer type.

FIG. 2. Structural changes upon activation of Ni₃Si₂O₅(OH)₄ shown by (a) x-ray diffraction (XRD) and (b) Pair Distribution Function (PDF) data. The PDF show the atom-atom correlations within a material allowing determination of the local structure of the material. The local structure information provided by PDF clearly shows an increase in unsaturated metal sites.

FIG. 3. FTIR spectra of the as-prepared (unactivated) and activated catalyst. Note the splitting of hydroxyl peak around 3650 cm⁻¹, which is a signature of the active site for oxidation.

FIG. 4. XPS spectra of the Si 2p, O 1s, and Ni 2p emission lines of pristine (unactivated) and activated catalysts.

FIG. 5. X-ray pair distribution function data for as-prepared Ni₃Si₂O₅(OH)₄ (circles) and fit (black line) (A). X-ray pair distribution function data for activated Ni₃Si₂O₅(OH)₄₋ₓ (yellow circles) and fit (black line) (B). The fitting data correspond to the models shown in FIGS. 6 and 7.

FIG. 6. A schematic representation of the as-prepared Ni₃Si₂O₅(OH)₄ material. Fully coordinated M (Ni) sites are shown in black, undercoordinated M (Ni) sites are shown in white, and the X (Si) position is shown in dark grey. The as-prepared model from the PDF shows the presence of unsaturated metal sites that correspond to the edges of the material.

FIG. 7. A schematic representation of the activated Ni₃Si₂O₅(OH)₄₋ₓ material as determined by fit to the PDF data. Fully coordinated M (Ni) sites are shown in black, undercoordinated M (Ni) sites are shown in white, and the X (Si) position is shown in dark grey. The activated model from the PDF show the number of unsaturated metal sites approximately doubles.

### DETAILED DESCRIPTION

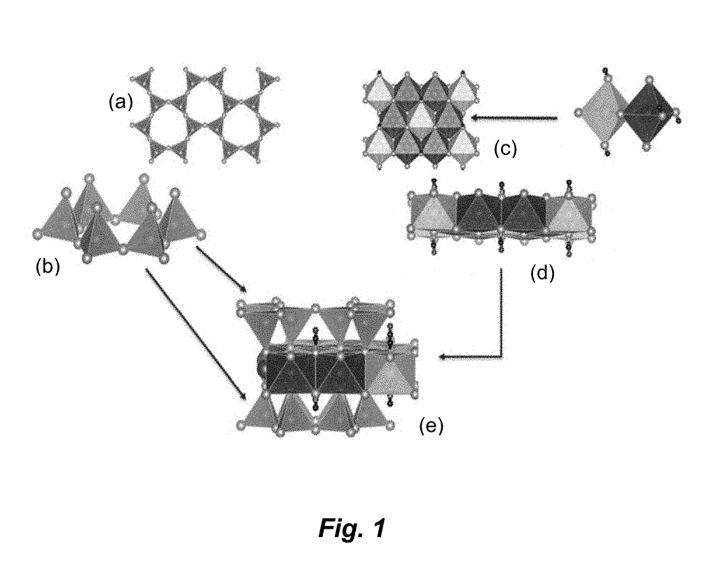

A heterogeneous phyllosilicate catalyst capable of oxidizing primary and secondary alcohols has been developed. Owing to their novel structure and highly tunable compositions, phyllosilicates are a promising new family of catalysts. Phyllosilicates are complex silicates minerals composed of alternating layers of corner-sharing SiO₄ tetrahedra and edge-sharing MO₄(OH)₂ metal layers. Phyllosilicates are subdivided into two major groups, 1:1 phyllosilicates, which contain one tetrahedral layer for every octahedral layer and 2:1 phyllosilicates which contain two tetrahedral layers for every octahedral layer (FIG. 1).

The phyllosilicate structure is accompanied by an interlayer space that may be filled with a neutral species or interlayer cations if the structure needs to be charge balanced. Within this structure there are five unique sites that can be substituted to tune the catalytic properties: the tetrahedral (T) site, the trans-coordinated octahedral site (Oₜ) in yellow (corners of item (c) in FIG. 1), the cis-coordinated octahedral site (O_{c}) in red (sites directly internal to the Oₜ sites), the interlayer species (I), and hydroxyl site (H).

Traditionally, this type of material has been used as an acid-base catalyst. However, the 1:1 phyllosilicate Ni₃Si₂O₅(OH)₄ has recently shown promise for oxidation reactions, transforming glycerol into value added products such as lactic acid. This material can be prepared by low-temperature hydrothermal methods by dissolving stoichiometric ratios of precursors in a sealed vessel in water at 200° C. Heating the resulting material at 500° C. results in the loss of protons creating catalytically active sites on the surface. However, an accurate understanding of the local coordination environment of the oxidatively active sites is needed to derive the mechanism for these reactions and is crucial to tuning their performance. Specifically, understanding the nature of the catalytically active sites within the structure is paramount for developing selectivity.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to bring about a recited effect, such as an amount necessary to form products in a reaction mixture. Determination of an effective amount is typically within the capacity of persons skilled in the art. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “phyllosilicate” catalyst refers to a catalyst composed of alternating layers of corner-sharing SiO₄ tetrahedra and edge-sharing MO₄(OH)₂ octahedra, where M is a suitable metal. Examples of suitable metals include Sc, Ti, V, Cr, Mn, Fe, Co, Ni, Cu, Zn, and Al, where the catalyst comprises M moieties in the 2+ oxidation state and/or M moieties in the 3+ oxidation state.

The term “1:1 phyllosilicate catalyst” refers to a phyllosilicate catalyst that contains one SiO₄ tetrahedral layer for each MO₂(OH)₄ octahedral layer.

The term “2:1 phyllosilicate catalyst” refers to a phyllosilicate catalyst that contains two SiO₄ tetrahedral layers for each MO₄(OH)₂ octahedral layer.

The term “heterogeneous catalyst” refers to a catalyst that occupies a different phase from the reactants, and optionally, the products (e.g., solid catalyst, liquid reactants).

The term “selective” as used herein refers to the preferential oxidation of one functional group in the presence of other functional groups, or the oxidation of a functional group in favor of a different functional group.

### Embodiments of the Invention

Phyllosilicates can be prepared and activated to provide catalysts for the oxidation of organic compounds that include at least one aliphatic alcohol. The oxidation occurs in the presence of oxygen and does not require other oxidants such as hydrogen peroxide. Accordingly, the invention provides methods for oxidizing alcohols without the use of hydrogen peroxide or other oxidants other than molecular oxygen. When activated, a proton, an entire hydroxyl group, or both, are liberated from the structure of the phyllosilicate, creating a material with a mixed-valence state that can be used for oxidizing alcohols, for example, to aldehydes or ketones.

The invention thus provides a heterogeneous catalyst comprising a crystallized phyllosilicate structure and mixed oxidation states of one or more metals in the phyllosilicate structure, wherein the catalyst is in an activated catalyst form such that protons, hydroxyls, or both, have been removed from the surface of the catalyst compared to the non-activated catalyst form. Furthermore, the phyllosilicate catalysts described herein do not have interlayer carbonates, and typically lack carbonate moieties altogether. Because the activated catalyst form is stable at 20-30° C., the catalyst can be isolated and stored for days or months while retaining its catalytic activity. After used for an oxidation reaction, the catalyst can be recovered, e.g., by filtration, and reused.

In one embodiment, the phyllosilicate structure has the formula M₂X₄O₁₀(OH)₂.4H₂O; AM₂X₄O₁₀(OH)₂.4H₂O; M₃X₂O₅(OH)₄; M₂X₂O₅(OH)₄; or A₂M₃X₄O₁₀(OH)₈; wherein

M is Sc, Ti, V, Cr, Mn, Fe, Co, Ni, Cu, Zn, Mg or Al, or a solid solution of any two or more of the recited elements, wherein the catalyst comprises M moieties in the 2+ oxidation state, in the 3+ oxidation state, or M moieties in the 2+ oxidation state and other M moieties in the 3+ oxidation state;

A is Li, Na, K, Rb, Cs, NH₄, Mg, Ca, Sr, or Ba; or a solid solution of any two or more of the recited elements, and

X is Fe, Al, Si, or Ti, or a solid solution of any two or more of the recited elements.

Solid solutions of two or more metals can be readily prepared by adjusting the ratio of metal salts used in the phyllosilicate preparation, as would be readily recognized by one of skill in the art.

In various embodiments, phyllosilicates of the following formulas can be prepared and then activated to provide activated catalysts of the invention. In one embodiment, the as-prepared phyllosilicate has the formula:

M₂X₄O₁₀(OH).4H₂O

wherein M is one or more of Sc, Ti, V, Cr, Mn, Fe, Co, Ni, Cu, Zn, and Al in the 2+ or 3+ oxidation state, and X is Fe, Al, Si, or Ti.

In another embodiment, the phyllosilicate catalyst has the formula:

AM₂X₄O₁₀(OH).4H₂O

wherein M is one or more of Sc, Ti, V, Cr, Mn, Fe, Co, Ni, Cu, Zn, and Al in the 2+ or 3+ oxidation state, A is Li, Na, K, Rb, Cs, NH₄, Mg, Ca, Sr, or Ba, and X is Fe, Al, Si, or Ti.

In another embodiment, the phyllosilicate catalyst has the formula:

M₃X₂O₅(OH)₄

wherein M is one or more of Sc, Ti, V, Cr, Mn, Fe, Co, Ni, Cu, Zn, Al in the 2+ or 3+ oxidation state, and X is Fe, Al, Si, or Ti. One specific example of a catalyst having the formula M₃X₂O₅(OH)₄ is [Ni_{2.4}Zn_{0.6}]Si₂O₅(OH)₄, where M is thus a solid solution of Ni and Zn.

In another embodiment, the phyllosilicate catalyst has the formula:

M₂X₂O₅(OH)₄

wherein M is one or more of Sc, Ti, V, Cr, Mn, Fe, Co, Ni, Cu, Zn, and Al in the 2+ or 3+ oxidation state, and X is Fe, Al, Si, or Ti.

In yet another embodiment, the phyllosilicate catalyst has the formula:

A₂M₃X₄O₁₀(OH)₈

wherein M is one or more of Sc, Ti, V, Cr, Mn, Fe, Co, Ni, Cu, Zn, and Al in the 2+ or 3+ oxidation state, A is Li, Na, K, Rb, Cs, NH₄, Mg, Ca, Sr, or Ba, and X is Fe, Al, Si, or Ti.

In one specific embodiment, M is Ni. In various embodiments, Ni is the only metal in the phyllosilicate catalyst. In other embodiments, the phyllosilicate catalyst comprises Ni and Zn.

Hydrothermal methods afford highly crystalline powders and gives tight control over compositions that can be prepared. Examples include KCo₃Si₄O₁₀(OH)₂, K(Co,Zn)₃Si₄O₁₀(OH)₂, and KZn_{2.6}Si₄O₁₀(OH)₂, showing that the metal and metal solid solution can be varied simply by varying the amounts of metal salts used to prepare the composition.

Examples of useful 2:1 phyllosilicates include KFe₃Si_{3.5}O₁₀(OH)₂, KCo₃Si₄O₁₀(OH)₂, and KZn_{2.6}Si₄O₁₀(OH)₂. Substituted 2:1 phyllosilicates can also be prepared, such as K[Zn_{(2.6-x)}Niₓ]{Si₄}O₁₀(OH)₂, K[Zn_{(2.6-x)}Coₓ]{Si₄}O₁₀(OH)₂, and K(Zn_{(2.6-x)}Feₓ){Si_{(4-y)}Fe_{y}}O₁₀(OH)₂.

Examples of useful 1:1 phyllosilicates include Ni₃Si₂O₅(OH)₄, Mg₃Si₂O₅(OH)₄, and Co₃Si₂O₅(OH)₄. Substituted 1:1 phyllosilicates can also be prepared, such as [Ni₃]{Si₍₂₋ₓ₎Alₓ}O₅(OH)₄, [Ni₍₃₋ₓ₎Mgₓ]{Si₂}O₅(OH)₄, [Ni₍₃₋ₓ₎Feₓ]{Si₍₂₋ₓ₎Alₓ}O₅(OH)₄, [Ni₍₃₋ₓ₎Znₓ]{Si₂}O₅(OH)₄, [Ni₍₃₋ₓ₎Coₓ]{Si₂}O₅(OH)₄, and [Ni₍₃₋ₓ₎Feₓ]{Si₂}O₅(OH)₄.

In various embodiments, the catalyst comprises phyllosilicate tetrahedral sheets, octahedral sheets, or 2:1 tetrahedral:octahedral layers. In certain embodiments, the catalyst comprises platelet structures.

In one specific embodiment, the catalyst comprises Ni₃Si₂O₅(OH)₄₋ₓ, where x is about 0.5 to about 3. Once activated, any of the phyllosilicate catalysts described herein can be shown by modifying its formula with “−x” after the number of (OH) groups in the formula, where x is about 0.5 to about 3.

By activating the phyllosilicates, a catalyst can be prepared wherein the ratio of OH:O ions on catalyst surface is less than 1:2. In other embodiments, a catalyst can be prepared wherein the ratio of OH:O ions on catalyst surface is less than 1:1.

The invention also provides a method to oxidize an alcohol moiety of an organic compound comprising contacting an organic compound that comprises an alcohol moiety with an effective amount of a heterogeneous catalyst described herein, for example,

a heterogeneous catalyst, wherein the heterogeneous catalyst comprises a crystallized phyllosilicate structure and mixed oxidation states of one or more metals in the phyllosilicate structure, wherein the catalyst is in an activated catalyst form such that protons, hydroxyls, or both, have been removed from the surface of the catalyst compared to the non-activated catalyst form; and

a base; in the presence of oxygen;

at a temperature sufficient to oxidize the alcohol moiety;

thereby providing an oxidized organic compound.

The alcohol moiety can be oxidized to an aldehyde, ketone, or carboxylic acid. In some embodiments, the alcohol moiety can be selectively oxidized to an aldehyde.

The oxidation is carried out at any suitable and effective temperature. In one embodiment, the oxidation is carried out at a temperature of about 20° C. to about 120° C., about 20° C. to about 80° C., about 30° C. to about 120° C., about 30° C. to about 80° C., or at about any temperature up to about the boiling point of the alcohol-containing substrate.

The base used in the oxidation reaction can be an alkali metal hydroxide or an alkaline earth metal hydroxide.

The invention further provides a method to produce H₂ gas comprising contacting an organic compound that comprises a hydroxyl with an effective amount of a catalyst described herein, and a base, at a temperature sufficient to produce H₂ gas. The production of H₂ gas can be concurrent with dehydrogenation and optionally, subsequent decarboxylation, of the organic compound.

Substrate Scope. The oxidation methods described herein can be useful for oxidizing a hydroxyl moiety on a variety of organic substrates, including aryl, heteroaryl, and heterocyclic compounds that have aliphatic alcohol substituents, and polyols (e.g., diols, triols, tetrols, etc.) having 3 to 20 carbon atoms. Alcohols can be selectively oxidized to aldehydes by appropriate control of reaction conditions. Shorter chain aliphatic alcohols, e.g., (C₂-C₄)alkanols can also be oxidized, for example, to formates and acetates.

The invention yet further provides a method for preparing an oxidation catalyst comprising pre-calcining a phyllosilicate catalyst precursor (e.g., a pristine or as-prepared phyllosilicate) in a pre-calcining zone in air at a temperature of less than about 600° C. to form a partially deprotonated form of the phyllosilicate catalyst precursor, which form comprises mixed-valence states of metals in the phyllosilicate. The phyllosilicate catalyst precursor can be activated through thermal or chemical treatment to produce a composition with mixed oxidation states on the metals, which can act as a catalyst for oxidation reactions. The partial deprotonation includes the loss of a proton, the loss of a hydroxyl, or a combination thereof, from the surface of the oxidation catalyst.

In some embodiments, more than 20%, more than 25%, or more than 30% of protons attached to hydroxyls on the surface of the catalyst are removed by the pre-calcining. In various embodiments, the pre-calcining is carried out at a temperature of about 400° C. to about 590° C.

The invention also provides a method for preparing an oxidation catalyst, the method comprising: (a) preparing a transition-metal containing phyllosilicate catalyst precursor; and (b) pre-calcining the catalyst precursor in a pre-calcining zone in air at a temperature of less than 600° C., thereby activating the transition-metal containing phyllosilicate to form a partially deprotonated form of the phyllosilicate catalyst precursor.

The following Example is intended to illustrate the above invention and should not be construed as to narrow its scope. One skilled in the art will readily recognize that the Example suggests many other ways in which the invention could be practiced. It should be understood that numerous variations and modifications may be made while remaining within the scope of the invention.

### Example 1. Phyllosilicate Clay Catalyst Preparation and Catalytic Activity

Reagents.

Metasilicic acid (H₂SiO₃), nickel nitrate hexahydrate (Ni(NO₃)₂.6H₂O), sodium hydroxide (NaOH), potassium hydroxide (KOH), glycerol (C₃H₈O₃), benzyl alcohol (C₇H₈O), cinnamyl alcohol (C₉H₁₀O), and O₂ were used as received.

Materials Synthesis.

Metasilicic acid (H₂SiO₃) was added to 15 mL of distilled water and stirred under moderate heat until fully dissolved. The resulting clear solution was cooled to room temperature (20-23° C.) and nickel nitrate (Ni(NO₃)₂.6H₂O) was added under constant stirring until dissolved, at which point the solution was gelled by the addition of NaOH. This gel was allowed to sit overnight (optionally 1-4 days) before transferring to a Teflon lined stainless steel autoclave and heated to 200° C. for 24-36 hours. The resulting teal powder was collected by vacuum filtration, washed with distilled water, and dried under vacuum to form the pre-catalyst. The pre-catalyst was then activated by heating at 500° C. for 6 hours in air to provide the catalyst. On activation, the pristine powder changes color to an ashy black, which is a reflection of a partial change in oxidation from Ni²⁺ to Ni₃₊. Composition and phase were confirmed by inductively coupled plasma-optical emission spectroscopy (ICP-OES) and phase verified by powder x-ray diffraction (XRD).

Other catalysts can be prepared by replacing the nickel nitrate (Ni(NO₃)₂.6H₂O) with a different metal salt. Useful metal salts include nitrates, acetates, sulfates, and halides, at any level of hydration. Metal salts such as MZ_{b}.(0-9)H₂O where M is a metal M as described herein, Z is nitrate, acetate, sulfate, or halide, and the b subscript after Z is an integer that matches the oxidation state of the metal M, can also be used to prepare the oxidation catalysts. Specific examples include NiCl₂.6H₂O, Fe^{II}(SO₄).7H₂O, Fe(NO₃)₃.9H₂O, MgCl₂, Mg(NO₃)₂.6H₂O, Zn(NO₃)₂.6H₂O, CoCl₂.6H₂O, Co(NO₃)₂.6H₂O, each of which have been used in the preparation of phyllosilicates and were found to be successful when used as activated catalysts.

Catalysis.

Oxidation can be performed in a three-neck flask equipped with a reflux condenser, filled with air at ambient pressure or in a Fisher-Porter bottle (FPB) at 383 K (110° C.) pressurized at either 50 or 95 psi O₂. The general reaction procedure was the following: the catalyst and substrate were transferred to a flask and the reaction was initiated by the addition of NaOH. The following reaction conditions were used for all reactions unless stated otherwise: 50 mg of Ni₃Si₂O₅(OH)₄ activated catalyst, 5 mL of alcohol, and 0.5M-0.8M NaOH, heated to 348 (75° C.) K with pure O₂ in the headspace, with mechanical stirring (300 rpm).

Density Functional Theory Calculations.

First principles calculations were performed in the Vienna Ab Initio Simulation Package (VASP) (Kresse et al., Phys. Rev. B 54, 11169 (15 Oct. 1996), within the projector augmented wave formalism (Blochl, Phys. Rev. B 50, 17953 (15 Dec. 1994), and the PBEsol functional (Perdew et al., Phys. Rev. Lett. 100, 136406 (4 Apr. 2008); a cutoff energy of 500 eV was used. To account for van der Waals interactions the D3 correction of Grimme was applied (J. Chem. Phys. 132, 154104 (2010)). For calculations in which defects are introduced at the clay surface it is necessary to correct for the well-known lack of localization of electrons within DFT; we have employed the DFT+U approach, where a parameter (U) penalizes partial occupancy of orbitals, thereby correcting for the self-interaction error in DFT and reinforcing charge localization (Dudarev et al., Phys. Rev. B 57, 1505 (15 Jan. 1998). A U value of 5 eV for Ni is employed, as in previous calculations of Ni based systems (Bengone et al., Comp. Mat. Sci. 24(1-2), 192-198 (May 2002)). The slab models used to simulate the surface have a macroscopic dipole across the unit cell due to the lack of inversion symmetry of the system normal to the surface; this can lead to divergence of electrostatic energy and spurious results. To correct for this in all cases final energies of slab systems had a dipole correction scheme applied to ensure accurate energies (Dubecký et al., J. Chem. Phys. 141, 094705 (2014)).

Model systems were constructed by initially taking the experimentally determined unit cell of the phyllosilicate material. This structure was then allowed to relax fully in terms of both unit cell parameters and ionic positions, using the settings outlined above and a k-point mesh sampling density with a target length cutoff of 25 Å, as prescribed by Moreno and Soler (Phys. Rev. B 45, 13891 (15 Jun. 1992)). The relaxed structure was then used to create a surface structure by cleaving the (001) surface, using three double layers as the slab thickness, with a 15 Å gap, which we have previously shown to be sufficient for obtaining converged electronic properties (Butler et al., Physical Review B: Condensed Matter and Materials Physics, 89 (11), 115320; Butler et al., Appl. Phys. Lett. 107, 231605 (2015)).

The surface was expanded to a (2×1) supercell. The possible effects of deprotonation and dehydroxylation were investigated by removing one hydrogen atom from the surface hydroxyls and one complete surface hydroxyl group respectively. The preferred adsorption geometry of glycerol was determined by initially considering the molecule in either standing or lying conformation, followed by a set of calculations of these configurations with the molecule transposed at regular spacing across the surface, sampling the entire surface with a resolution of 1 Å. A short molecular dynamics (MD) calculation, 5000 steps of 0.5 fs, at 400 K was then performed on the preferred configurations to allow the molecules to sample additional configurational space. The MD calculations were then cooled from 400 K to 0 K at a rate of 5×10⁻¹³ Ks⁻¹, which we have previously applied to generate accurate structures (Wahila et al., Chem. Mater., 2016, 28 (13), 4706-4713).

Results.

Structure.

The hydrothermal synthesis of Ni₃Si₂O₅(OH)₄ has been reported previously. To prepare the starting material, metasilicic acid (H₂SiO₃) was added to 15 mL distilled water and stirred under moderate heat until fully dissolved. The resulting clear solution was cooled to room temperature and nickel nitrate (Ni(NO₃)₂.6H₂O) was added under constant stirring until dissolved, at which point the solution was gelled by the addition of NaOH. This gel was allowed to sit overnight before transferring to a Teflon lined stainless steel autoclave and heated to 200° C. for 24-36 hours. The resulting teal powder was collected by vacuum filtration, washed with distilled water and dried under vacuum. The material was activated for catalysis by heating at 500° C. for 6 hours in air. On activation, the pristine teal powder changes color to black reflecting the chance in oxidation from Ni²⁺ to Ni³⁺.

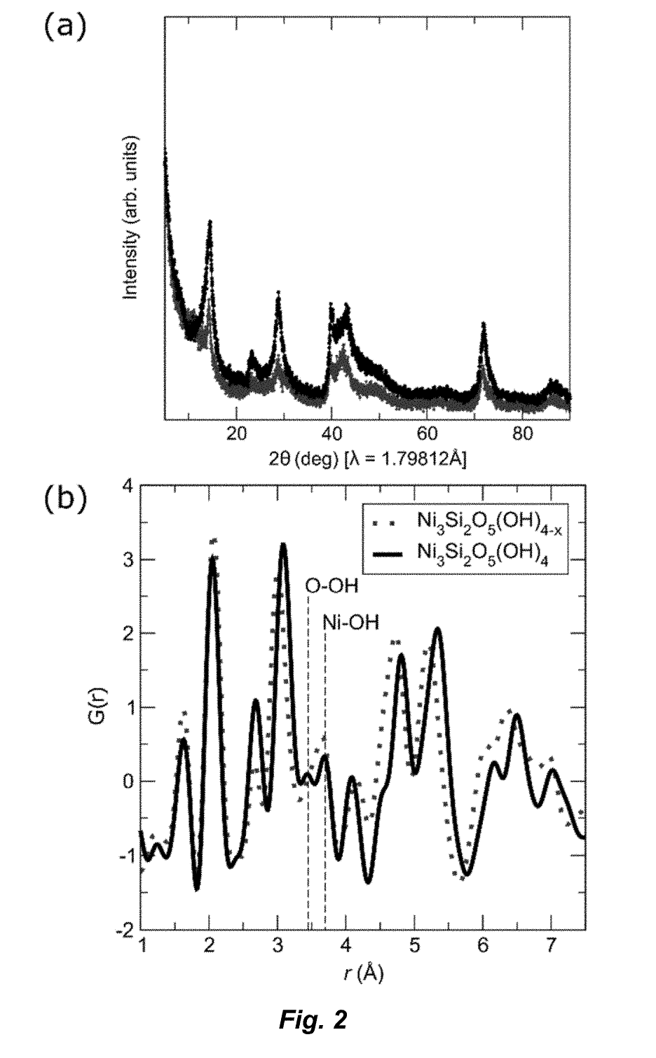

FIG. 2 shows the powder X-ray diffraction pattern for pristine (red; lower line) and activated (black; upper line) Ni₃Si₂O₅(OH)₄, which match well with the nickel serpentine mineral pecoraite (JCPDS 49-1859). The most prominent change upon activation is a loss of intensity on the (001) peaks, presumably as a result of delamination of the sheets as residual interlayer water is removed. To further elucidate the structural changes associated with catalytic activation, total scattering neutron data was collected and pair distribution function analysis was performed. The pristine and activated patterns, while similar, show clear changes in the interatomic distances associated with the oxygen ion in the SiO₄ tetrahedron and the hydroxyl ions. These changes correspond to the loss of protons from the parent Ni₃Si₂O₅(OH)₄ to produce a new composition, Ni₃Si₂O₅(OH)₄₋ₓ, where the loss of protons is compensated through the oxidation of Ni²⁺ to Ni³⁺.

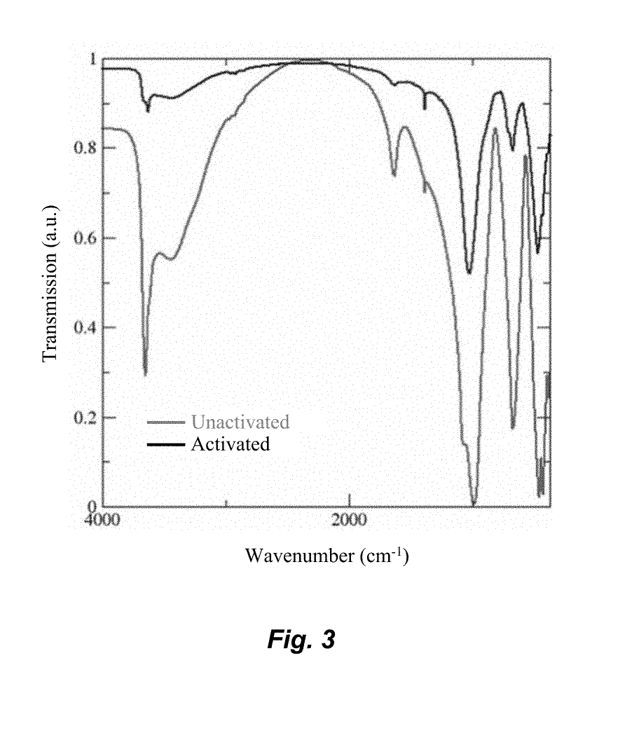

The FTIR spectra (FIG. 3) of pristine Ni₃Si₂O₅(OH)₄ shows the expected absorption for morphological platelets of Ni₃Si₂O₅(OH)₄ and these features are retained upon activation. Ni—O_{stretch} 3650 cm⁻¹, O—H_{stretch} 3438 cm⁻¹, Si—O_{stretch} 1073, 1000, and 982 cm⁻¹, Si—O—Ni_{stretch} 669 cm⁻¹, and Si—O_{bend} 455 and 427 cm⁻¹. Significant loss of intensity in region 3650-3440 cm⁻¹ indicates the loss of protons upon activation. The diminished δH₂O bending mode (1632 cm⁻¹) indicates the loss of water adsorbed on the surface or between layers upon activation. This loss of water as well as protons results in the delamination of sheets in the structure. The growth of a high frequency shoulder in the Ni—O_{stretch} region indicates a morphological change from platelets to nanotubes upon activation. The composite band in the region 980-1075 cm⁻¹ is also strongly affected by loss of hydrogen or hydroxyls weakening hydrogen bonding forces between layers. Another significant distinction is in the range of 500-750 cm⁻¹ corresponding to Si—O—Ni stretching vibrations. The doublet that forms at 427 and 455 cm⁻¹ is unique to nickel containing chrysotile compounds indicating the unique influence of nickel on the bending vibrations of the Si—O layer. The observed differences in the shape and position of the IR absorption bands can be used to distinguish the result of dehydration processes across the surface creating a new nickel environment.

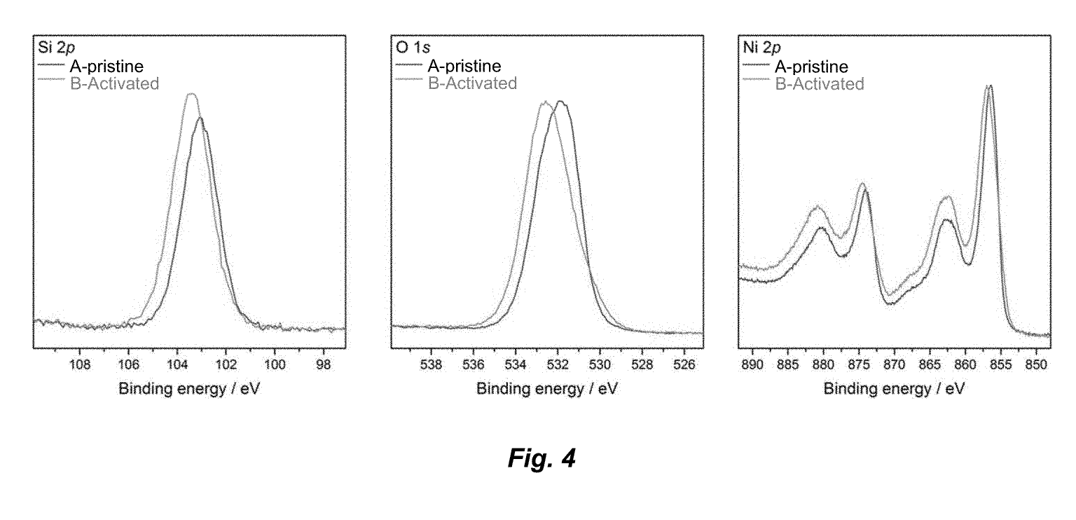

To determine the local surface environment and oxidation state of nickel, X-ray photoelectron spectroscopy was performed on the pristine and activated samples. As can be seen in FIG. 4, clear changes in the oxygen 1s binding energy, indicating the ratio of OH:O ions on the surface shifts from 61:39 in the pristine sample to 32:68 in the activated sample and indicating that ˜30% of the protons attached to the hydroxyl were lost during activation. This is further reflected in the Si 2p states, which shift to high energy after activation, indicating an increase in the prevalence of silicate groups compared to hydroxyls. Using the Ni 2p lines it is difficult to discriminate the exact oxidation state, but the activated phase does show a shift to higher energy, consistent with oxidation to the trivalent state.

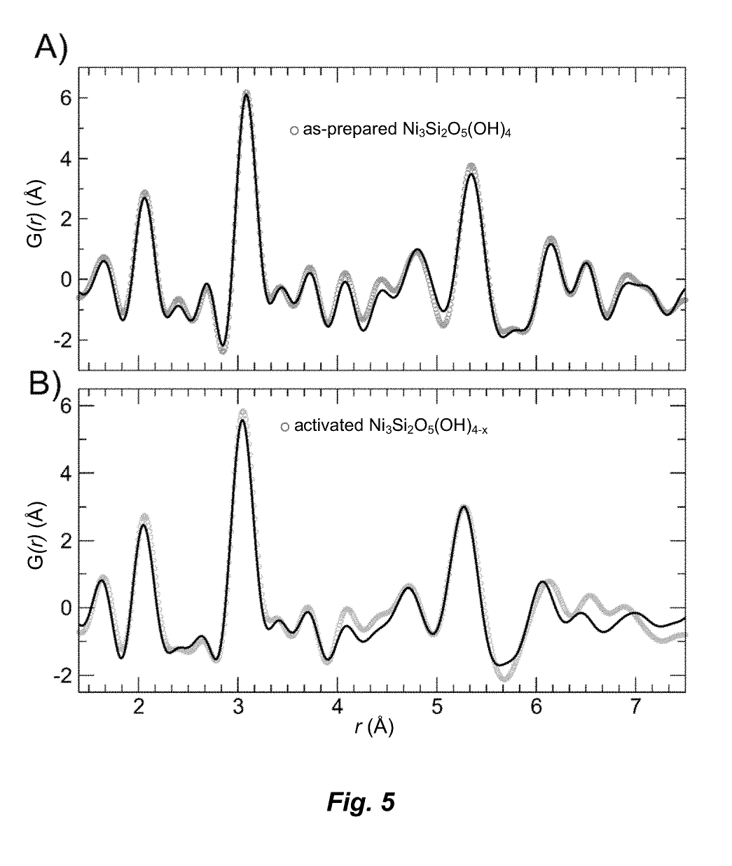

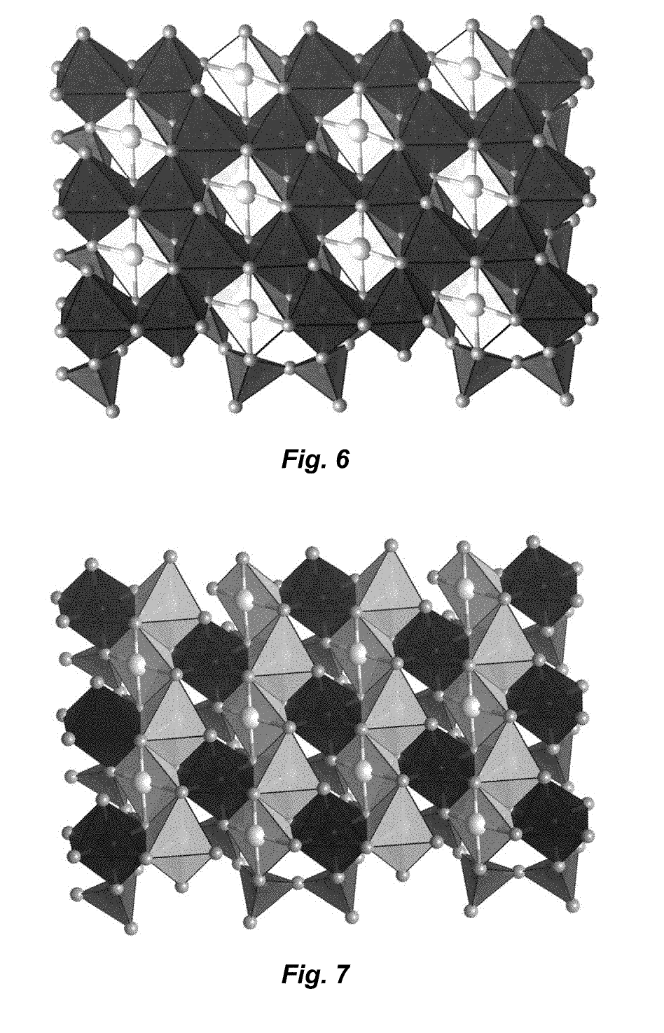

The X-ray pair distribution function demonstrates the atom-atom correlations within the structure of a molecule or material. It can be used to determine the local environment of a unit cell, informing understanding of active site structure for catalysis. As shown in FIG. 5, the X-ray PDF indicates that the average Si—O bond length is about 1.61, consistent with cristobalite (Dove, M., Keen, D., Hannon, A. et al. Phys Chem Min (1997) 24: 311. https://doi.org/10.1007/s002690050043). The appearance of strain in phyllosilicates is often caused by a size mismatch between the shared oxygen of the silicate tetrahedra and the metal octahedra. The silicate tetrahedra form hexagonal rings that have a certain degree of flexibility, and therefore, can distort to accommodate the lattice difference between the octahedral and tetrahedral layers. Distorting the silica ring and or the hydroxide layer may lead to extended defects such as bending of sheets, puckering of layers or intergrowths. Often this strain cannot be modeled appropriately by diffraction techniques and misappropriated to a lengthening in the Si—O bond length. Here size mismatch is accommodated by a buckling or rocking in the Silicate ring. This is an important structural influence on the microenvironment of the phyllosilicate surface.

Pair distribution function also indicates that the average Ni—O bond length is 2.05 and remains that length upon activation. A second Ni—O bond length can be seen at 2.4 angstroms (FIG. 5), and a corresponding O—O distance at 2.7 angstroms. Activation results in a loss of intensity at 2.4 and 2.7 associated with a loss of oxygen or hydroxyls. Activation results in diminished intensity at the 2.4 and 2.7 peaks indicating a loss of oxygen. This results in the remaining Ni—O bonds hybridizing further and their energy being lowered into the bandgap of the material, resulting in a black color. It is believed that these are the result of 5-coordinate Nickel sites created by the loss of OH, resembling uncompensated edge sites, but now on the surface.

Neutron PDF can be used to further elucidate structural changes associated with catalytic activation. The pristine and activated patterns, while similar, show clear changes in the interatomic distances associated with the oxygen ion in the SiO₄ tetrahedron and the hydroxyl ions. This change is associated with the loss of protons and activation of Ni₃Si₂O₅(OH)₄ is only seen in the short-range order further indicating structure retention upon activation. The increased peak intensity seen in FIG. 5 corresponds to the O—H and Ni—OH distance in pristine and activated Ni₃Si₂O₅(OH)₄ respectively. As can be seen in FIG. 5, the intensity increases in the activated samples corresponding to a decrease in the negatively scattering H⁺.

The fit for the pristine pair distribution function corresponds to a platelet morphology silicate with some 5 coordinate Ni Sites, and the fit for the activated pair distribution function corresponds to 30-60% more active sites. X-ray photoelectron spectroscopy confirms that the number of lost hydroxyls is around 30-60%.

### Example 2. Oxidation of Benzyl Alcohol Using Phyllosilicate Clay Catalysts

The activated catalyst can oxidize primary alcohols such as benzyl alcohol to its corresponding aldehyde using molecular oxygen as the only oxidant. The catalyst can be an activated form of a serpentine-type phyllosilicate of the formula M₃X₂O₅(OH)₄, such as N_{3-y}M^{a}_{y}Si₂O₅(OH)₄₋ₓ where N is Ni; M^{a} is Sc, Ti, V, Cr, Mn, Fe, Co, Cu, Zn, Mg or Al, y is about 1, 2, 3, or any fraction in between any two of the recited integers, and x is 1, 2, 3, or any fraction in between any two of the recited integers. In this experiment, the experiment was conducted with each of activated Ni₃Si₂O₅(OH)₄₋ₓ, activated Ni_{2.5}Zn_{0.5}Si₂O₅(OH)₄₋ₓ, and activated Ni_{1.8}Mg_{1.2}Si₂O₅(OH)₄₋ₓ. See Scheme 1.

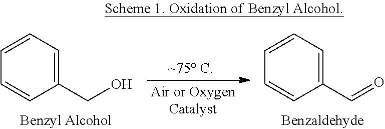 [SMILES: OCc1ccccc1 ; O=Cc1ccccc1]

Oxidation of benzyl alcohol can be performed (A) in a three-neck flask, filled with air or O₂ at ambient or balloon pressure or (B) in a Fisher-Porter Bottle (FPB) at 348 K (75° C.) pressurized at either 50 or 95 psi air or O₂. The general reaction procedure was as follows: the activated catalyst (0.05 g) and benzyl alcohol were transferred to the FPB and purged with 50 or 95 psi oxygen three times before the reaction was initiated. The oxidation of benzyl alcohol can be performed neat, or in the presence of polar or nonpolar solvents, as well as protic or aprotic solvents, such as toluene, acetonitrile and water. Either oxygen or air can be used as the oxidant. The air used was about ˜20% O₂ and ˜80% N₂. No reaction takes place in the presence of only nitrogen gas. The oxidation can take place between room temperature and 110° C. The reaction was run for 1 to about 12 hours, at which time most of the starting material was consumed.

The oxidation of benzyl alcohol to benzaldehyde was confirmed using ¹H NMR by the presence of the singlet resonance of the aldehyde proton at ˜10.03 ppm. This resonance shows no coupling because there are no neighboring protons. No evidence of the overoxidation product, benzoic acid (˜8.13 ppm), was observed in the ¹H NMR.

### Example 3. Oxidation of Cinnamyl Alcohol Using Phyllosilicate Clay Catalysts

The activated catalyst can oxidize primary alcohols such as cinnamyl alcohol to its corresponding aldehyde using molecular oxygen as the only oxidant. The catalyst can be an activated form of a serpentine-type phyllosilicate of the formula M₃X₂O₅(OH)₄, such as N_{3-y}M^{a}_{y}Si₂O₅(OH)₄₋ₓ where N is Ni; M^{a} is Sc, Ti, V, Cr, Mn, Fe, Co, Cu, Zn, Mg or Al, y is about 1, 2, 3, or any fraction in between any two of the recited integers, and x is 1, 2, 3, or any fraction in between any two of the recited integers. In this experiment, the catalyst was activated Ni₃Si₂O₅(OH)₄₋ₓ. See Scheme 2.

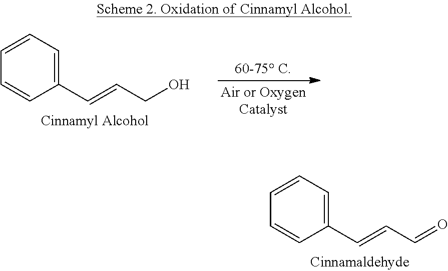 [SMILES: OC/C=C/c1ccccc1 ; O=C/C=C/c1ccccc1]

The oxidation was performed in a Fisher-Porter Bottle (FPB) at 333 K (60° C.) pressurized at 50 psi O₂. The general reaction procedure was as follows: the catalyst (0.05 g) and cinnamyl alcohol (3 mL, neat) were transferred to the FPB and purged with 50 psi oxygen three times before the reaction was initiated at 60° C. The reaction was run for 1 to about 12 hours.

The oxidation of cinnamyl alcohol to cinnamaldehyde was confirmed by the resonance of the aldehyde proton doublet at ˜9.68 ppm in its ¹H NMR spectrum. The retention of the double bond is confirmed by the presence of a doublet rather than a singlet due to two conformations of the double bond. The proton resonance is coupled to the neighboring proton (6.63 ppm), which was observed in its ¹H-¹H cos y NMR spectrum. A small amount of overoxidation product, cinnamic acid, was observed by the presence of a proton peak at ˜7.83 ppm coupled to its neighboring proton at ˜7.59 ppm as seen in the ¹H-¹H cos y NMR spectrum. The occurrence of the overoxidized product can be reduced by addition of a solvent to the reaction conditions.

### Example 4. Oxidation of Glycerol Using Phyllosilicate Clay Catalysts

The activated catalyst can oxidize polyols such as glycerol to lactic acid at an elevated temperature using a base and molecular oxygen as the only oxidant. The catalyst can be an activated form of a serpentine-type phyllosilicate of the formula M₃X₂O₅(OH)₄, such as N_{3-y}M^{a}_{y}Si₂O₅(OH)₄₋ₓ where N is Ni; M^{a} is Sc, Ti, V, Cr, Mn, Fe, Co, Cu, Zn, Mg or Al, y is about 1, 2, 3, or any fraction in between any two of the recited integers, and x is 1, 2, 3, or any fraction in between any two of the recited integers. This experiment was successfully carried out using each of the activated Ni₃Si₂O₅(OH)₄₋ₓ, activated Ni_{2.7}Zn_{0.3}Si₂O₅(OH)₄₋ₓ, activated Ni_{2.5}Zn_{0.5}Si₂O₅(OH)₄₋ₓ, activated Ni_{1.5}Mg_{1.5}Si₂O₅(OH)₄₋ₓ, and activated Ni_{2.2}Mg_{0.8}Si₂O₅(OH)₄₋ₓ catalysts. See Scheme 3.

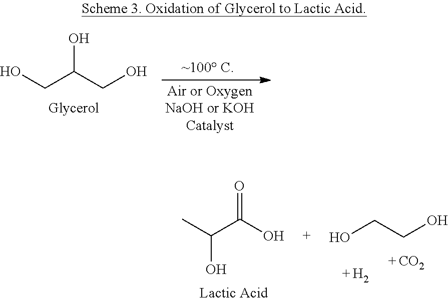

Oxidation of glycerol can be performed (A) in a three-neck flask equipped with a reflux condenser, filled with air at ambient pressure or (B) in a Fisher-Porter bottle (FPB) at 383 K (110° C.) pressurized at either 50 or 95 psi O₂.

The general reaction procedure was the following: the catalyst (0.05 g) and glycerol (0.3 g/L, 5 mL) and sodium hydroxide (NaOH, 0.5M-8M) were transferred to FPB and purged with oxygen three times before the reaction was initiated. Either oxygen or air can be used as the oxidant. In the presence of only nitrogen gas glycerol is transformed to ethylene glycol. In the absence of base, no lactic acid is created. The oxidation takes place between 60 and 210° C. The oxidation of glycerol to lactic acid can be performed neat, or in the presence of polar or nonpolar solvents, as well as protic or aprotic solvents, such as toluene, acetonitrile and water. The reaction was run for 1 to about 12 hours. A mechanism for the oxidation of glycerol to lactic acid is shown in Scheme 4.

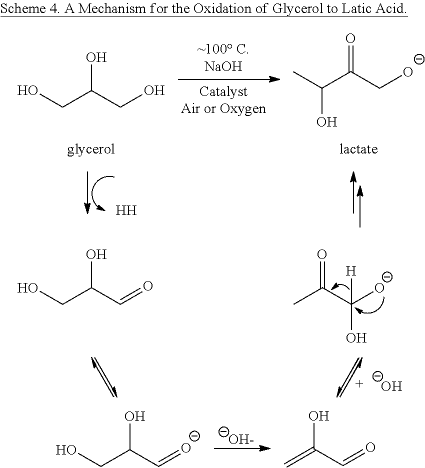

The oxidation of glycerol to lactic acid was confirmed by a quartet resonance at ˜3.96 ppm and ˜1.17 ppm in its ¹H NMR spectrum. The appearance of lactic acid is further corroborated with the creation of H₂ gas, which was observed in a gas injection gas chromatogram obtained while conducting the experiment. Small amounts of ethylene glycol and carbon dioxide are created as byproducts in this oxidation. The ratio of products can be controlled by the amount of base and oxygen pressure/content, where ethylene glycol increases with base concentration, and lactic acid production increases with oxygen pressure.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A heterogeneous catalyst comprising a crystallized phyllosilicate structure and mixed oxidation states of one or more metals in the phyllosilicate structure, wherein the catalyst is in an activated catalyst form such that protons, hydroxyls, or both, have been removed from the surface of the catalyst compared to the non-activated catalyst form, to provide an activated catalyst form, and the activated catalyst form is stable at 20-30° C.

2. The catalyst of claim 1 wherein the phyllosilicate structure has the formula M₂X₄O₁₀(OH)₂.4H₂O; AM₂X₄O₁₀(OH)₂.4H₂O; M₃X₂O₅(OH)₄; M₂X₂O₅(OH)₄; or A₂M₃X₄O₁₀(OH)₈; wherein
M is Sc, Ti, V, Cr, Mn, Fe, Co, Ni, Cu, Zn, Mg or Al, or a solid solution of any two or more of the recited elements, wherein the catalyst comprises M moieties in the 2+ oxidation state, in the 3+ oxidation state, or M moieties in the 2+ oxidation state and other M moieties in the 3+ oxidation state;
A is Li, Na, K, Rb, Cs, NH₄, Mg, Ca, Sr, or Ba; or a solid solution of any two or more of the recited elements, and
X is Fe, Al, Si, or Ti, or a solid solution of any two or more of the recited elements.

3. The catalyst of claim 2 wherein M is Ni.

4. The catalyst of claim 2 wherein the catalyst comprises Ni and Zn.

5. The catalyst of claim 2 wherein the catalyst comprises phyllosilicate tetrahedral sheets, octahedral sheets, 1:1 tetrahedral:octahedral layers, or 2:1 tetrahedral:octahedral layers.

6. The catalyst of claim 2 wherein the catalyst comprises Ni₃Si₂O₅(OH)₄₋ₓ, where x is about 0.5 to about 3.

7. The catalyst of claim 2 wherein the ratio of OH:O ions on catalyst surface is less than 1:2.

8. The catalyst of claim 2 wherein the ratio of OH:O ions on catalyst surface is less than 1:1.

9. The catalyst of claim 2 wherein the catalyst comprises platelet structures.

10. A method to oxidize an alcohol moiety of an organic compound comprising contacting an organic compound that comprises an alcohol moiety with an effective amount of:
a heterogeneous catalyst, wherein the heterogeneous catalyst comprises a crystallized phyllosilicate structure and mixed oxidation states of one or more metals in the phyllosilicate structure, wherein the catalyst is in an activated catalyst form such that protons, hydroxyls, or both, have been removed from the surface of the catalyst compared to the non-activated catalyst form; and
a base; in the presence of oxygen;
at a temperature sufficient to oxidize the alcohol moiety;
thereby providing an oxidized organic compound.

11. The method of claim 10 wherein the alcohol moiety is oxidized to an aldehyde, ketone, or carboxylic acid.

12. The method of claim 10 wherein the oxidation is carried out at a temperature of about 20° C. to about 120° C.

13. The method of claim 10 wherein the base is an alkali metal hydroxide or an alkaline earth metal hydroxide.

14. A method to produce H₂ gas comprising contacting an organic compound that comprises a hydroxyl with an effective amount of a catalyst of claim 1, and a base, at a temperature sufficient to produce H₂ gas.

15. The method of claim 14 wherein the production of H₂ gas is concurrent with dehydrogenation and optionally, subsequent decarboxylation, of the organic compound.

16. A method for preparing the catalyst of claim 1 comprising pre-calcining a phyllosilicate catalyst precursor in a pre-calcining zone in air at a temperature of less than about 600° C. to form a partially deprotonated form of the phyllosilicate catalyst precursor, which form comprises mixed-valence states of metals in the phyllosilicate.

17. The method of claim 16 wherein the phyllosilicate catalyst precursor is activated through thermal or chemical treatment to produce a composition with mixed oxidation states on the metals.

18. The method of claim 16 wherein the partial deprotonation comprises the loss of a proton, the loss of a hydroxyl, or a combination thereof, from the surface of the oxidation catalyst.

19. The catalyst of claim 16 wherein more than 20% of protons attached to hydroxyls on the surface of the catalyst are removed by the pre-calcining.

20. The method of claim 16 wherein the pre-calcining is carried out at a temperature of about 400° C. to about 590° C.

21. A method for preparing an oxidation catalyst, the method comprising: (a) preparing a transition-metal containing phyllosilicate catalyst precursor; and (b) pre-calcining the catalyst precursor in a pre-calcining zone in air at a temperature of less than 600° C., thereby activating the transition-metal containing phyllosilicate to form a partially deprotonated form of the phyllosilicate catalyst precursor.
